(19) United States
(12) Patent Application Publication
(10) Pub. No.: US 2024/0336966 A1
(43) Pub. Date: Oct. 10, 2024

(54) INTEGRATED OPTICAL REAL-TIME MOLECULE SEQUENCING VIA PHOTONIC CRYSTAL LASERS/DETECTORS

(71) Applicant: Seagate Technology LLC, Fremont, CA (US)

(72) Inventors: Vivek Krishnamurthy, Edina, MN (US); Aditya Jain, Minneapolis, MN (US); Gemma Mendonsa, Minneapolis, MN (US); Anil J. Reddy, Minneapolis, MN (US)

(21) Appl. No.: 18/607,991

(22) Filed: Mar. 18, 2024

Related U.S. Application Data
(60) Provisional application No. 63/458,278, filed on Apr. 10, 2023.

(51) Int. Cl. C12Q 1/6869

(52) U.S. Cl. CPC C12Q 1/6869

(57) ABSTRACT

A laser-based molecular sequencing system includes a two-dimensional photonic crystal surface emitting laser (PCSEL) array including a plurality of PCSEL devices located in a first layer, each PCSEL device oriented in a direction perpendicular to the first layer. The system also includes a controller configured to modulate a bias on at least one PCSEL device of the plurality of PCSEL devices, the modulation configured to selectively operate each PCSEL device as an emitter or detector at a point in time based on the bias.

### CROSS-REFERENCE TO RELATED APPLICATIONS

This application claims the benefit of U.S. Provisional Patent Application having Ser. No. 63/458,278 titled “INTEGRATED OPTICAL REAL-TIME MOLECULE SEQUENCING VIA PHOTONIC CRYSTAL LASERS/DETECTORS” filed Apr. 10, 2023, the entire contents of which are incorporated herein by reference for all purposes herein.

### SUMMARY

In general, this case is directed to utilizing photonic crystal surface emitting lasers (PCSELs) for sequencing molecules, such as nucleotides of DNA, RNA, and/or various proteins. The present disclosure provides various integrated optical chip embodiments that enable laser and photodetector integration. The result is reduced cost and complexity, while also providing optical field enhancement for sequencing, via both labeled and label-free techniques, all in real-time. Further benefits include opportunities for parallelization and scalability.

According to a first aspect of the present disclosure, a laser-based molecular sequencing system is disclosed. According to the first embodiment, the system includes a two-dimensional photonic crystal surface emitting laser (PCSEL) array including a plurality of PCSEL devices located in a first layer, each PCSEL device oriented in a direction perpendicular to the first layer. The system also includes a controller configured to modulate a bias on at least one PCSEL device of the plurality of PCSEL devices, the modulation configured to selectively operate each PCSEL device as an emitter or detector at a point in time based on the bias.

According to a second aspect of the present disclosure, a method of performing laser-based molecular sequencing is disclosed. According to the second aspect, the method includes receiving a sample molecule to be sequenced. The method also includes modulating a first bias to a PCSEL device to cause the PCSEL device to emit an output signal, where the output signal interacts with the sample molecule. The method also includes modulating a second bias to the PCSEL device to cause the PCSEL device to operate as a detector. The method also includes receiving an input signal at the PCSEL device based on the output signal interacting with the sample molecule. The method also includes analyzing the sample molecule based on the input signal.

According to a third aspect of the present disclosure, a computer program product for performing laser-based molecular sequencing is disclosed. According to the third aspect, the computer program product includes a computer-readable storage medium having program code embodied therewith, the program code including computer-readable program code configured to cause a processor to perform steps. According to the third aspect, the steps include receiving a sample molecule to be sequenced. The steps also include modulating a first bias to a PCSEL device to cause the PCSEL device to emit an output signal, where the output signal interacts with the sample molecule. The steps also include modulating a second bias to the PCSEL device to cause the PCSEL device to operate as a detector. The steps also include receiving an input signal at the PCSEL device based on the output signal interacting with the sample molecule. The steps also include analyzing the sample molecule based on the input signal.

These and various other features and advantages will be apparent from a reading of the following detailed description.

### BRIEF DESCRIPTION OF THE DRAWINGS

The present invention will be further explained with reference to the appended Figures, wherein like structure is referred to by like numerals throughout the several views, and wherein:

FIGS. 1A and 1B show cross-section views of components of an example photonic crystal surface emitting laser (PCSEL) system, according to various embodiments.

FIG. 2 shows a photonic crystal for use with a PCSEL, according to various embodiments.

FIG. 3 shows an example photonic bandgap for a PCSEL, according to various embodiments.

FIG. 4 graphically shows relative transmission and reflection efficiencies corresponding to photonic crystals, according to various embodiments.

FIG. 5 is a tabular representation of four nucleotides and possible signature signals, according to various embodiments.

FIG. 6 is a perspective view of an array of molecular wells/traps of a layer, as described herein.

FIGS. 7A and 7B schematically show methodologies of single molecule real-time sequencing in an integrated system using labeled and non-labeled DNA strands, according to various embodiments.

FIG. 8 shows strong optical fields at the base of a metal well/trap, according to various embodiments.

FIG. 9 shows a process, according to various embodiments.

FIG. 10 is a block schematic diagram of a computer system according to embodiments of the present disclosure.

### DETAILED DESCRIPTION

The methods, systems, and features described herein provide for utilizing semiconductor lasers including photonic crystal surface-emitting lasers (PCSELs), and include embodiments applicable to optical sequencing of molecules including DNA, RNA, and protein, and more particularly to employing PCSELs to provide real-time DNA, RNA, and protein molecular sequencing that can be used with various strands, either labeled or label-free, with benefits including reduced complexity, energy efficiency, cost effectiveness, parallelization, high speed, among other benefits.

Current DNA sequencing methods face many limitations. Example limitations include sequence read length, sensitivity, run time, and cost. A higher sensitivity or signal/noise ratio can improve sequencing accuracy in long reads. The length of a DNA strand to be sequenced can also be limited when labels are used, as most labels do not give a sufficiently strong signal and require multiple molecules to generate signals simultaneously. As the sequence length increases, the individual molecular signals tend to fall out of sync, limiting the length of accurate sequence. Run times are often long due to the need to pause after each base incorporation to obtain an optical signal and/or remove the labels. Cost is also high due to the need of labels and bulky optical tools for the sequencing.

Existing commercial optical sequencing methods utilize labeled (or tagged) nucleotides for fluorescence detection. Certain methods require expensive and bulky optical instruments, to couple the light from a laser and to detect the fluorescence signature from the sample area. Typically, this equipment includes a separate laser source, a separate detector, and a separate zero waveguide chip, which are not typically integrated into the same chip. Hence, the methods depend on the aspects of the particular instrumentation.

Certain existing and experimental laser-based systems, including certain complex optical arrangements including silicon photonics chips, have additional limitations. Previous proposals used laser and waveguides with branch to deliver optical signal to DNA traps. In these arrangements, power from one source is distributed to thousands of sequencers, through waveguiding, filtering, etc.

With PCSELs photonic crystal emitters/detectors discussed herein, power distribution, waveguiding, and filtering can all be avoided. Instead, each plasmonic sequencer is addressed by a photonic crystal emitter/detector, optionally within a chip, allowing parallelization and fast sequencing.

To address shortcoming in existing methods and systems, provided herein are methods and systems that utilize PCSELs for stimulation and emission to bypass the need for bulky optical instruments and to provide a low-cost and portable solution for DNA, RNA, and protein sequencing. The methods can use labeled or label-free molecules. Complete on-chip integration makes it compact and low cost and thus having a much wider commercialization possibility as a portable system for DNA, RNA, and protein sequencing.

The methods and systems of this disclosure preferably utilize a PCSEL (semiconductor) chip having a dielectric layer and a metal layer on top. As discussed herein, the metal layer has molecular traps (e.g., DNA traps) as a sample chamber with one or more associated PCSEL device that serves a dual purpose of stimulation and detection depending on an applied electrical bias. When in detection mode, the PCSEL device can detect a labelled/non-labelled nucleotide's spectral signature, in the form of intensity, wavelength response, time constant, binding kinetics, and/or combination thereof. The PCSELs chip, may not need integration with a spectrometer and/or filter, providing packaging cost savings and benefits of simplicity, among other benefits.

The present disclosure provides a significant breakthrough in laser and optical sequencing applications and technologies by utilizing PCSELs in place of existing complex optical arrangements. PCSELs, as described herein, have been shown to simplify and to improve both sequencing scalability and efficiency limitations. Embodiments have the benefit of small emitters, which can be <2 microns [um] wide, and also benefit from coherent laser beam locking, due to laterally-confined lasing modes. Smaller emitters (which can double as detectors/sensors, as described below) can be enabled through smaller active areas of PCSELs, as described further, below. Thus, PCSELs avoid a need for external lasers/detectors where the PCSELs directly interact with the plasmonic wells/traps (e.g., on top) as described herein. The compact PCSEL size, together with non-requirement of an external laser, enables high optical sequencing density and energy efficiency when using PCSELs in sequencing environments, such as for DNA, RNA, or protein sequencing. Also, PCSELs can utilize high modulation frequencies, for characterizing time constants for sequencing.

As discussed herein, each emitter of the PCSELs can be assigned to a particular well, or “trap.” By so doing, existing setups that include complex optical guides can be completely avoided, making parallelization and other improvements possible.

In general, and according to various embodiments of the photonic crystal and PCSELs contemplated herein, PCSELs allow for a smaller separation of laser emitters/detectors, thus more laser/detector channels, and thus facilitate massively parallel optical sequencing operations. Hence, using PCSELs has the benefits of significant savings in terms of wafer real-estate and laser-chip packaging costs, since there is no longer a need for beam and optical collimation structures.

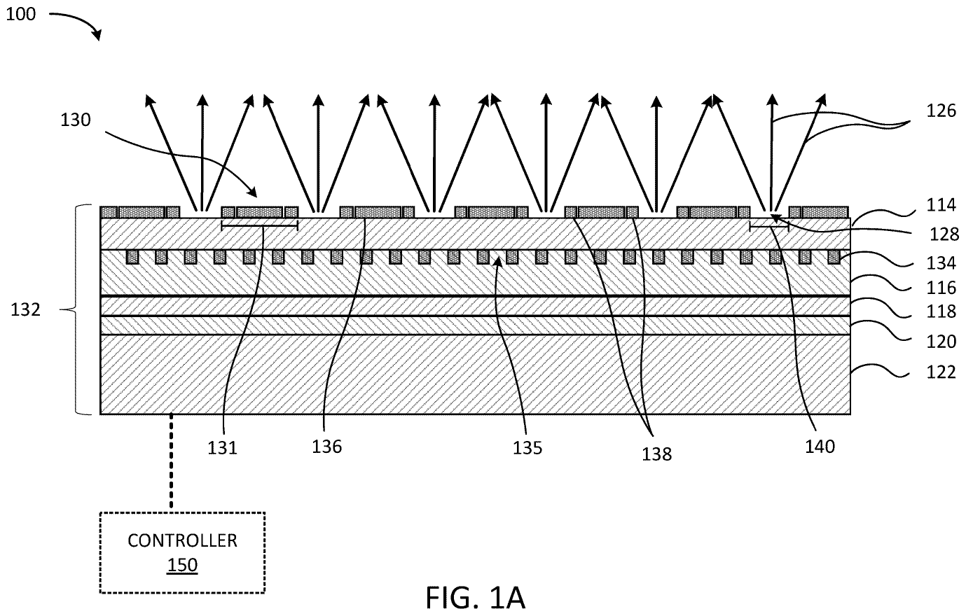

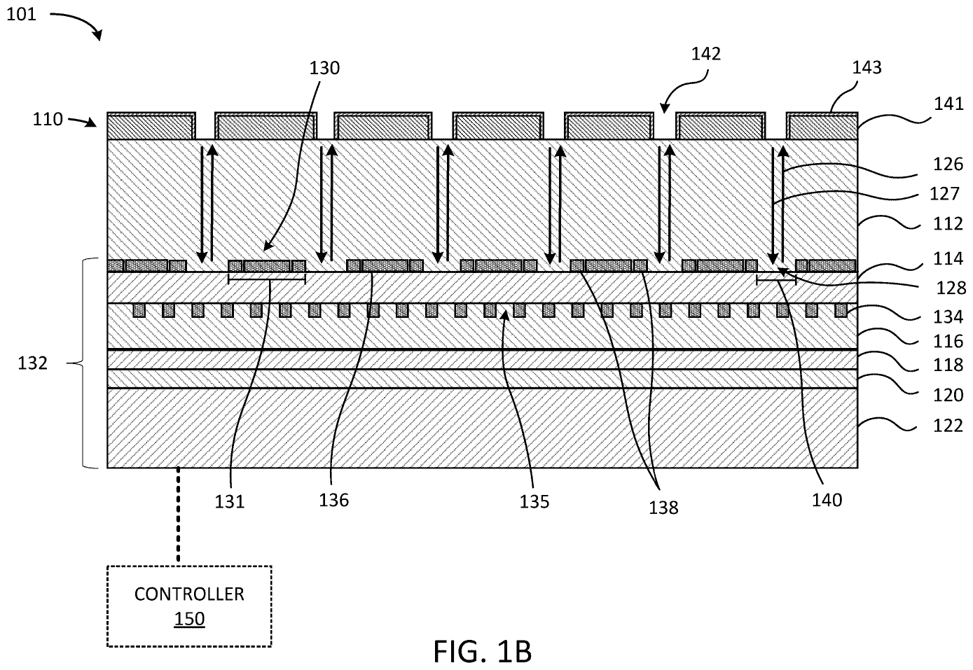

Turning now to FIGS. 1A and 1B, FIG. 1A shows a cross section view of a system 100 including components 132 of an example photonic crystal surface emitting laser (PCSEL) device operatively connected to a controller 150. As contemplated herein, the example PCSEL system 100 includes an array of emitters/detectors 128, in which optical emission of coherent beams can be electronically controlled. PCSELs represent a significant technological breakthrough in semiconductor lasers, and have many and diverse useful applications, according to various embodiments, herein. System 101 of FIG. 1B adds molecular trap 142 features to system 100 of FIG. 1A. Throughout the following description it is understood that various embodiments can use systems 100 and 101 interchangeably where applicable.

As shown, the PCSEL system 100 includes a multi-layered, stacked structure in which multiple horizontal layers are stacked vertically, and which are configured to vertically and outwardly emit surface laser beam(s) 126 (or signals, more generally) from an end, such as an upper end, of system 101. As shown in FIG. 1B, the upper end of the system 101 can include a molecular trap array layer 110 as shown in the embodiment of system 101. The molecular trap array layer 110 can be external or integrated according to various embodiments. As shown one or more molecular traps 142 (or plasmonic wells) within and separated by metal layer portion 141 (e.g., aluminum or the like), preferably coated with a passivated layer 143, which can together form one or more raised barrier that can be associated with the layer 110 and traps 142.

Still referring to FIG. 1B, below molecular trap array layer 110 is a dielectric layer 112, optionally a low-index dielectric layer comprising, e.g., Al₂O₃ or SiO₂. The dielectric layer 112 can be approximately 10 nm to 10 um thick and can be deposited in various embodiments. In other embodiments, layer 112 can be patterned to focus the beam from 128 to 143. In other embodiments, the dielectric layer 112 can include multiple layers, including open space or air. The dielectric layer 112 can be between 100 nm and 100 um in height/thickness in various embodiments.

At the base of and below the dielectric layer 112 as shown, is a sub-portion of the system stack including a number of PCSEL components 132 as described and shown in FIG. 1A. At an upper portion of the PCSEL layer 132 are electrical contacts 136 for controlling coupling between emitters in gaps and electrical contacts 138 for controlling laser emission (and detection) at contacts. Electric contacts 136 and 138 can be coplanar, as shown, and contacts 138 can be circular around a gap 140.

An overall contact width of groups 131 of two contacts 138 and one contact 136 as shown can have a contact width 130 of approximately 1-100 um. Contacts 138 can provide for forward and reverse PCSEL bias control, as discussed further below. As shown, between adjacent groups 130 can be an open gap 140 (emitter 128) between contacts 138 of a gap width of approximately 0.1-10 um, or more preferably, approximately 0.5-5 um in gap width. Note that FIGS. 1A and 1B, for clarity, may not be drawn to scale, and that gap 140 may preferably be substantially larger than trap 142 width (“d” as shown) described herein. As described herein, laser emissions of laser beam(s) 126 can enter the dielectric layer 112 through gap(s) (at emitter 128), which as described in greater detail below, are representative of PCSEL emitters/detectors 128. Electrical isolation may be needed between multiple emitters/detectors 128 (with common ground), since emitters/detectors 128 would be used for emission with forward bias and photodetection (as a detector) with reverse bias.

As used herein, emitters/detectors 128 can be referred to as any of a) emitters, b) detectors, or c) both. It is to be understood that the PCSEL device acting as an emitter, detector, or both is a device that is selectively configured to have aspects of emission, detection, or both, depending on a (e.g., electrical or other operative) bias or control applied thereto. It is understood that these devices can be referred to herein interchangeable as emitters 128, detectors 128, or the like depending on a desired function and bias.

The PCSEL components 132 also include a p-doped layer 114. The p-doped layer 114 can be about 1-2 um in height/thickness in various embodiments. Located below the p-doped layer 114 (e.g., AlGaAs) as shown is a p-doped separately confined heterostructure (SCH) layer 116 (e.g., InGaP). The p-doped SCH layer 116 can be about 50 nm to 1 um in height/thickness in various embodiments.

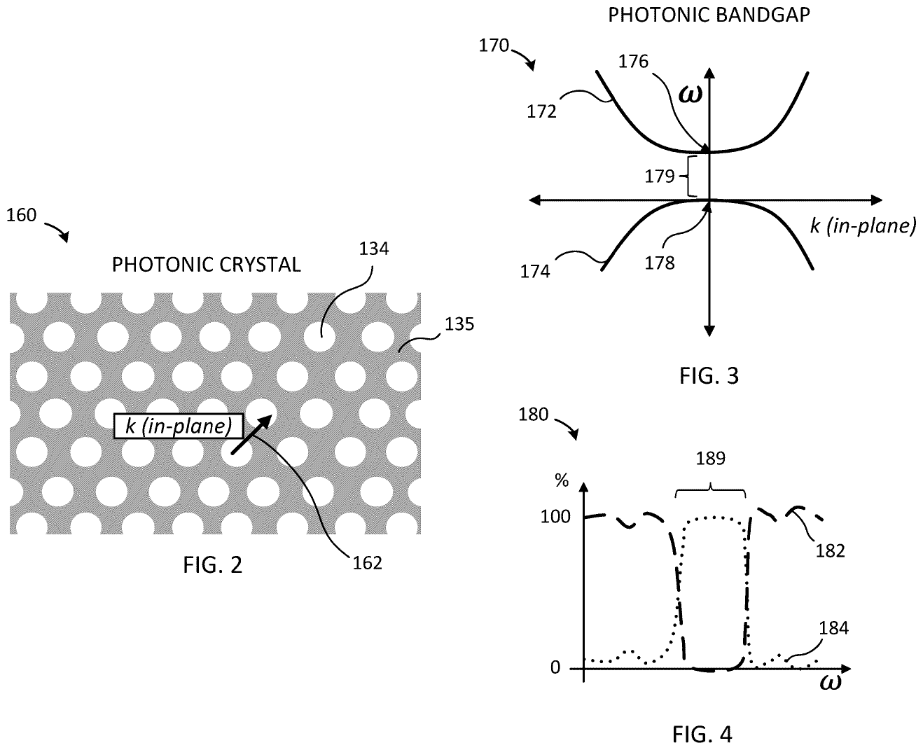

As shown, layer 116 can comprise photonic crystals (in two-dimensional lattice, see also 160 of FIG. 2) comprising relatively low refractive index material (or holes) 134 with corresponding gaps 135 (e.g., of higher reflective index material) located therebetween. The low refractive index material lattice of holes 134 can either be partially or fully etched through p-SCH layer 116. In various embodiments, the photonic crystal holes 134 cause the excitation of band-edge modes (see also FIG. 3) in the active layer 118. The low refractive index holes 134 can form a regular grid or pattern, and may or may not include one or more lattice irregularities also called “defects,” briefly discussed further below.

Located below the active p-doped SCH layer 116 in the stack of PCSEL components 132 of system 101 is a layer of multiple quantum wells/quantum dots (active elements) at 118. The layer 118 can be about 50 nm to 150 nm in height/thickness in various embodiments. Located below layer 118 is an n-doped SCH layer at 120 (e.g., AlGaAs). The n-doped SCH layer 120 can be about 50 nm to 1 um in height/thickness in various embodiments. Finally, at the base of PCSEL components 132 of system 101, as shown, is an n-doped substrate 122. The substrate 122 can be any suitable height/thickness in various embodiments. The layer arrangement and configuration presented in system 101 is to be viewed as an example, and other variations and modifications are also contemplated herein. For example, according to practical needs, number of n-doped, p-doped and/or active layers could be increased or decreased among other variations.

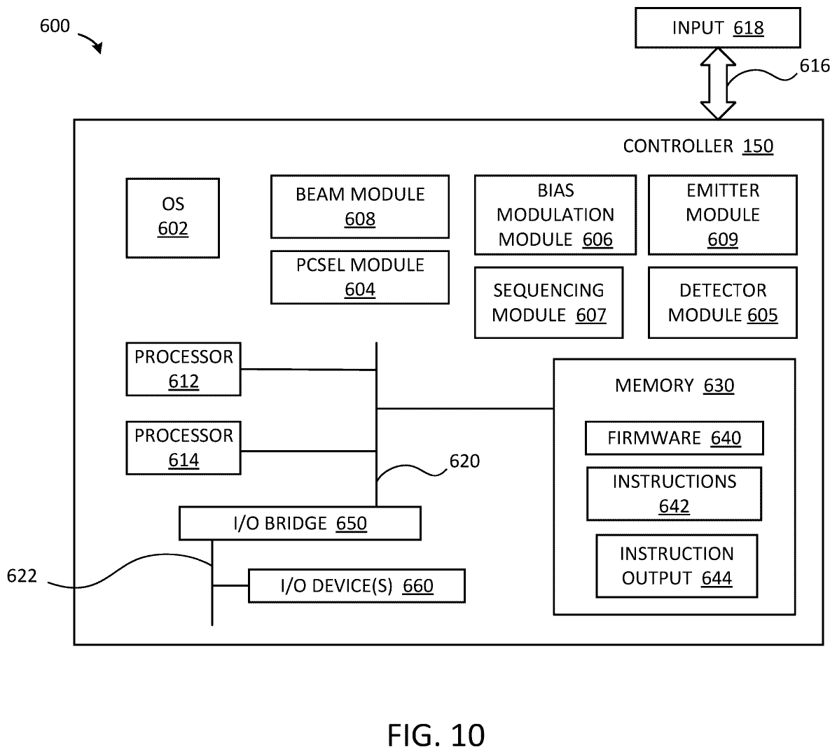

Shown operatively connected to the system 101 is an optional controller 150, an embodiment of which is described in greater detail in FIG. 10, below. The controller 150 optionally includes a bias (e.g., electrical) source or driver. The controller 150, as shown, can be operatively connected to the various components of system 101.

As used herein, the controller 150 can be configured to control the coupling at contacts 136 and 138 for electrical control of isolation between emitters and switching between forward and reverse bias for emission and photodetection respectively, such as for optical sequencing. For example, the controller 150 can be configured to control PCSEL emission and beam(s) 126, including but not limited to modulating a bias to PCSEL emitters/detectors 128 for operation and sequencing, as described herein. By using the controller 150 to control PCSEL emitters 128 and/or arrays thereof, various operations are achieved.

For example, the controller 150, by controlling forward and reverse bias at emitters/detectors 128, can achieve optical sequencing (e.g., using fluorescence) which can be detected using a PCSEL device also used as an emitter. As shown, the molecular trap array layer 110 can comprise one or more molecular traps 142 (optionally corresponding to a number of PCSEL devices, i.e., emitters/detectors 128).

The example photonic crystal lattice at layer 116, including lower refractive index areas 134 and higher refractive index areas 135 can take various shapes, arrangements, forms. One example arrangement of a photonic crystal layer 160 of FIG. 2, which shows a regular, two-dimensional, hexagonal lattice arrangement of lower refractive index holes 134 in the higher refractive index (and higher optical intensity) layer 135 of general photonic crystal layer 116 of the PCSEL structure of system 101. Also shown in a k (in-plane) wavevector 162 corresponding to a resulting emission. Layer 160 can represent a subset of layer 116, as described herein. In various other arrangements, some of the holes 134 could be removed/omitted to form “defects” in the lattice of layer 160. These defects, if present in higher refractive areas 135, can also be emission regions associated with emitters 128.

Reflection and transmission are contemplated in various components and embodiments, herein. In various embodiments, the lattice arrangement in layer 116 of photonic crystal lattice 160 formed by 134/135 is configured such that at least a mode has a distinctly lowest threshold pump power. In various embodiments, laser emission at emitters 128 can be controlled (e.g., using controller 150) using current injection. Furthermore, coupling between the emitters 128 can be controlled by controlling a loss (current injection) between emitters 128.

An example PCSEL photonic bandgap 179 is provided at graph 170 of FIG. 3. As shown, two bands 172 and 174 have band edges at relative minima 176 and maxima 178, respectively at a closest point between band edges, shown separated by bandgap 179. The two axes shown include k (in-plane) on the x-axis, and ω (angular frequency of light) on the y-axis. As used herein, k (in-plane) denotes a reciprocal of spatial periodicity of optical fields, or how many beats of periods are there in a unit distance along the plane.

Turning now to FIG. 4, graph 180 shows an example of corresponding photonic transmission 182 and reflection 184 percentages (efficiencies) 0-100%, at various angular frequencies ω and a corresponding band gap 189. Bandgap frequencies correspond to the region where in-plane reflection 184 is high and in-plane transmission 182 is low. Hence, with electrical pumping of optical region, in-plane photonic crystal modes in 134/135 are created and confined that are in-turn emitted through emitter 128. Since the mode is created by the photonic crystal the wavelength and phase are coherent. Hence the emission at all the emitters 128 is coherent. The above thus illustrates an operational principle of PCSELs.

As illustrated in FIGS. 3 and 4, various optical field distributions in photonic crystal lattices (e.g., layer 160) and resulting band structures are also contemplated. Wavevectors (k-in-plane 162, see FIG. 2) can be compared to spatial frequency (e.g., using formula ωa/2πc) to examine the photonic band gap and associated features and aspects. In some examples, no transverse electric (TE) modes may exist in the photonic band gaps shown at 179/189.

Examples of optical modes contemplated herein, are detailed in “Electronic control of coherence in a two-dimensional array of photonic crystal surface emitting lasers” to Taylor et al, and include a first-order TE mode, and a fundamental TE mode, although various other modes are also contemplated herein. As also contemplated herein, and by embedding photonic crystals 134/135 in a PCSEL device, the fundamental TE mode can be excited, by ensuring that they have the highest gain in the laser media. The resulting TE mode would be single mode, single frequency, or single wavelength. Modes contemplated are not only single mode and single frequency or single wavelength as they can be generated simultaneously over the entire crystal 160 comprising 134/135. Embedded crystals can thus allow for excitation of photonic crystal modes, including single or optionally multiple modes. Various modes can be coherent (same phase) as well, as discussed in Taylor. By having electronic control over the laser emission and detection, optical sequencing using PCSELs is achieved.

With the above structure and understanding of PCSELs, we turn now to employing PCSELs in optical sequencing system, and more specifically to biasing PCSEL devices to both optically emit and detect, as described in further detail below. A bias on the PCSELs can be modulated positive to negative, and thus each PCSELs device can be used for both detection and emission (of lasers), selectively. Based on stimulated emission detected at the PCSELs, detected phase, time decay, binding kinetics, and amplitude can be used to identify and sequence DNA, RNA, and/or protein nucleotides and various characteristics. Optionally, fluorescence and/or Raman signal is utilized.

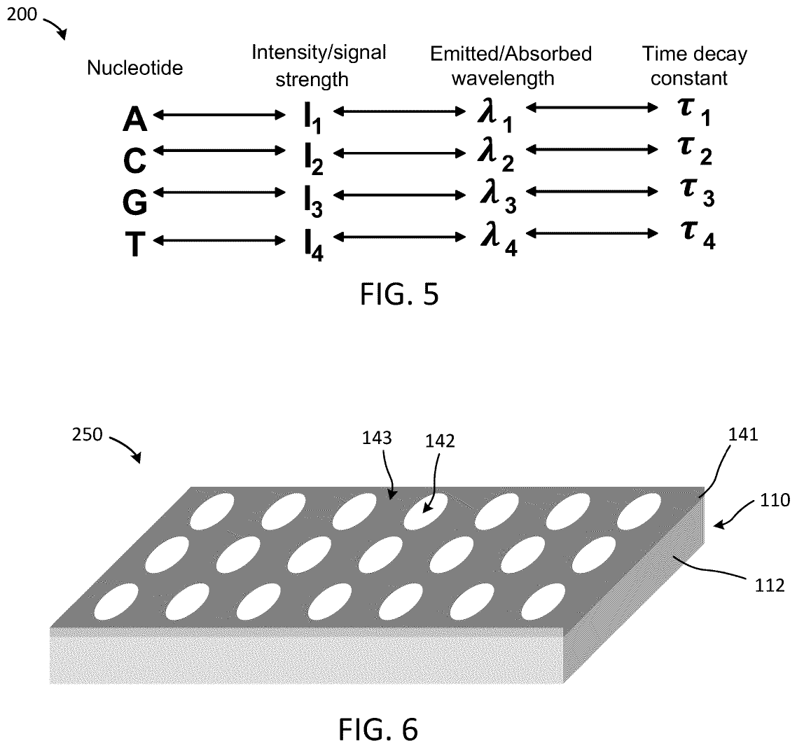

FIG. 5 is a tabular representation of four nucleotides and possible signature signals, according to various embodiments. Optical sequencing of DNA, RNA, and protein according to this disclosure involves providing the molecule to be sequenced to a sample area (referred to as a trap 142 herein) where an electromagnetic field is concentrated or enhanced, with the purpose of maximizing energy input into the sample molecule. The sample molecule emits energy that has some nucleotide-specific signature. Shown is a table listing the four nucleotides adenine (A), cytosine (C), guanine (G), and thymine (T) and their signature. The signature can be in signal intensity domain, wavelength domain, or time domain. A label or tag may facilitate distinguishing nucleotides in one or more of these output signatures. The following discussion provides various integrated sequencing systems that utilize these output signatures to identify the nucleotides and their order.

FIG. 6 is a perspective view of an array 250 of molecular traps 142 of a layer 110, as described herein. As shown, the traps 142 selectively break a passivated layer 143 surface of the metal portion 141 at respective openings. Passivated layer 143 preferably ensures that DNA, RNA, and/or protein molecules fall into the respective traps 142 with minimal friction or stick to the surface 143 and metal layer 141, e.g., by passivating the metal layer. The plasmonic trap 142 can be defined as an area in the metal portion 141 devoid of (metal) material; that is, the trap 142 can be a void in the layer 110 extending through to PCSELs components 132, which are provided below.

Although an example array of 3×7 traps 142 as shown, it is contemplated that hundreds to thousands to millions (or more) or any number of DNA traps 142 can be processed and positioned on top of PCSEL devices (emitters 128) for sequencing, as described herein.

The traps 142 can be configured to receive DNA, RNA, and/or protein molecules. In various preferred embodiments, the metal portion 141 of the molecular trap array layer 110 can be passivated so as to ensure single strand DNA/RNA/protein molecules, when introduced are not as likely to stick to the surface of the metal portion 141, but rather to provide for the DNA/RNA/protein molecules to fall into the traps 142 and become immobilized for PCSEL-based optical sequencing, as described below.

The metal portion 141 (e.g., aluminum layer, ˜100 nm thick) can optionally be non-stick for molecules being sequenced, and can be deposited on the dielectric layer 112, below. The metal portion 141 layer can be less than 500 nm in thickness, and can be provided with the traps 142 that can reach the dielectric layer 112, and the traps 142 can be of size ˜100 nm deep (h) and diameter (d) can be in the range of 30-300 nm.

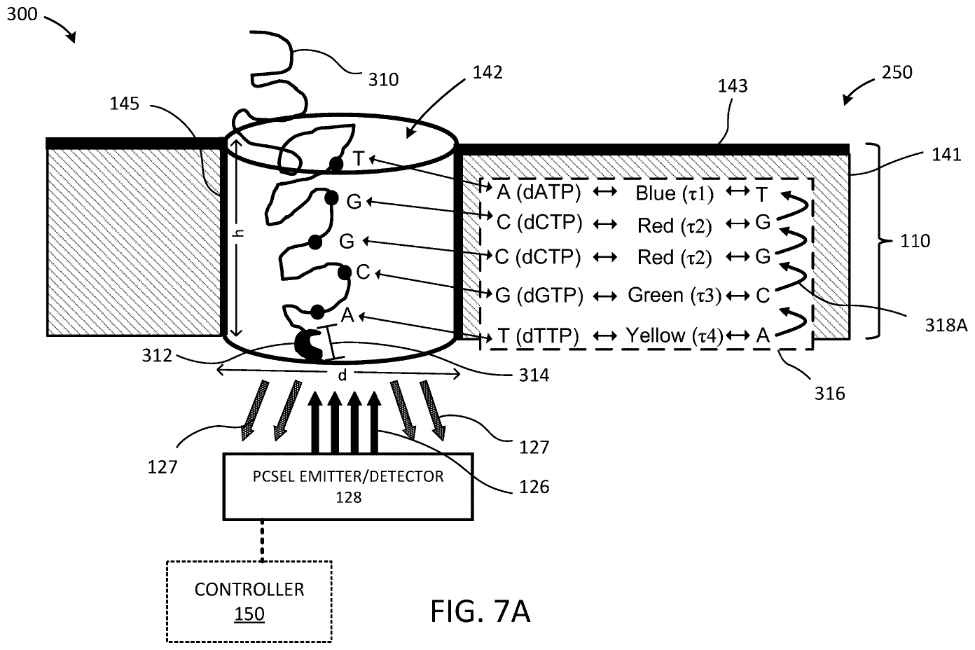

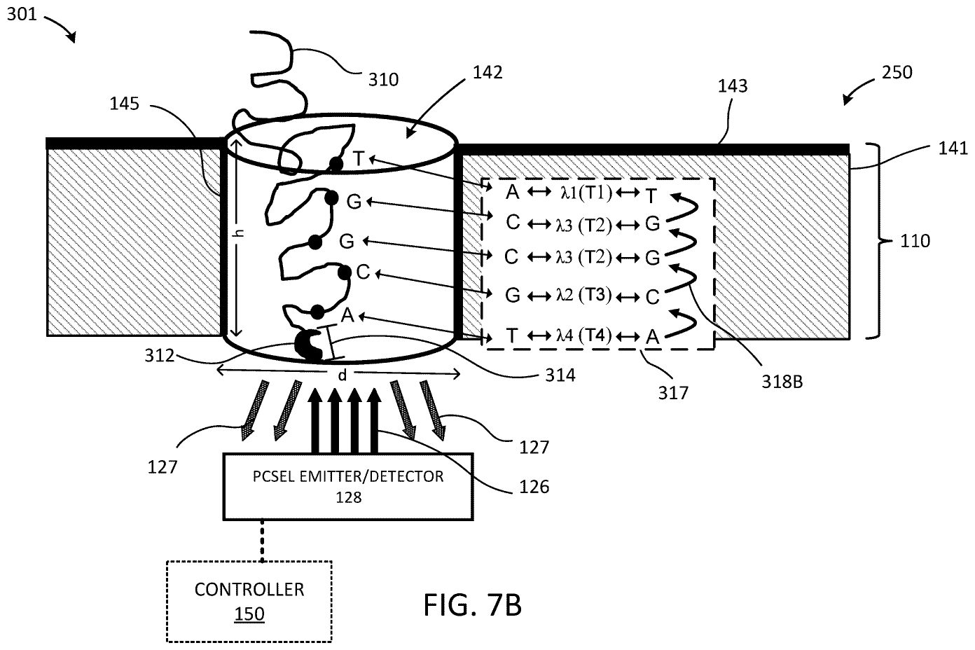

FIG. 7A schematically shows a methodology 300 of single molecule real-time sequencing in an integrated system. Methodology 300 uses fluorescence labeled or tagged free nucleotides and FIG. 7B shows a methodology 301 using untagged free nucleotides. In both figures, the methodologies utilize a similar trap 142 and PCSEL emitter 128. For simplicity, FIG. 7A will be first discussed, after which FIG. 7B will be discussed to the extent that it differs from FIG. 7A.

If DNA is to be sequenced, the molecule trap 142 may be called a DNA trap 142. If the molecule being sequenced is DNA, the trap can include a DNA polymerase 312, topoisomerase, or other DNA processing protein present, e.g., bound to a surface of the trap 142. In some embodiments, a surface of the trap 142 may be passivated, e.g., using PVPA (polyvinylphosphonic acid) to increase the likelihood of the polymerase and the DNA being immobilized on a surface of the trap 142, such as at the bottom of the trap 142. In some embodiments, the trap 142 has a width or diameter (d) as described above. Positioned in close proximity to the trap 142 is the PCSEL. Together, the trap 142 and the PCSEL components may be referred to collectively as the detector/sensor 128, or simply “PCSEL device,” but not necessarily so. By having multiple detectors 128 running simultaneously, the accuracy and/or rate of (optionally parallel) sequencing is increased. See also “Real time dynamic single-molecule protein sequencing on an integrated semiconductor device” Reed et al. (2022), which is hereby incorporated by reference herein for all purposes.

In operation, light from the emitters 128 enters the trap 142 from below as shown to interact with the molecule 310 (e.g., a DNA/RNA/protein strand, such as a single strand comprising nucleotides) in the trap 142. A specific spectral signature (e.g., wavelength) of light is emitted from the molecule 310. For DNA, the signature emitted is based on the complementary nucleotide being added to the single strand DNA by the polymerase 312. The emitted signature light is received at the PCSEL emitters (biased to operate as detectors) 128, and can be interpreted or analyzed using controller 150.

If the molecule 310 is DNA, each nucleotide of the DNA single strand can be characterized through single molecule real-time sequencing; the sequencing may be with either labeled or not-labeled nucleotides.

A single-strand DNA molecule 310 is shown in the trap 142. A DNA polymerase is present in the trap 142, as well as free (unattached) nucleotides (e.g., A, C, T, G). Although not necessary, the free nucleotides can be labeled or tagged. The DNA strand 310 is captured by the polymerase 312 and is primed for duplication. The appropriate complementary free nucleotide is paired to the template strand, one at a time after a time period (as described below).

The incorporation of the free nucleotide results in the emittance of a signal depending on which nucleotide (A, C, G, T) is incorporated to the strand. The PCSEL emitter (detector) 128 detects an output signal 127 from the molecule 310. The output signal 127 (described further below) can be a fluorescence signal (e.g., if labeled) or Raman signal, in the form of intensity, wavelength (color) and/or time decay.

Based on the detected signal 127, as the DNA moves through the detection region, a specific pattern of DNA sequence, associated with the specimen molecule, is generated by the controller 150 using the PCSEL emitter 128 as a sensor/detector.

The light emitted at the PCSEL emitter 128 interacts with the molecule 310 present in the trap 142. The incorporation of the free nucleotide results in the emittance of a signal 127 depending on which nucleotide (A, C, T, G) is incorporated to the strand 310. Depending on the specific signal 127, the nucleotide enables a specific response of the detector 128, thus identifying the intensity or wavelength and time decay of the signal 127 and correlating the signal 127 to a particular nucleotide. Based on the detected signal 127, as the DNA moves through the detection region, a specific pattern of nucleotide sequence is generated based on strand 310.

The PCSEL detector 128 can be used for real-time DNA sequencing, for example, with non-labeled Surface Enhanced Raman Spectroscopy (SERS) to identify the four DNA nucleotides (A, C, G, T) based on their unique Raman scattering spectra, in the form of intensity (or wavelength) response and/or time decay.

The molecule trap 142 as shown is located at an intersection of layer 110 and a beam 126 emitted from PCSEL emitter 128. The trap 142 has a volume optionally defined as the volume of a cylinder (e.g., volume=2πrh, where r=d/2; thus, volume can be expressed as: πdh), in this embodiment where the trap 142 is cylindrical, defined by a height (h) and a width or diameter (d). In some embodiments, the height (h) is about 100 nanometers and the trap diameter (d) as described above.

As shown, within the volume of the trap 142 is a DNA polymerase 312, optionally bounded to a wall or the floor of the trap 142. PVPA or another similar polymer may be present to facilitate adsorption of the polymerase 312/DNA 310 to the bottom of the trap 142; for example, the PVPA can enable adsorption to a fused silica surface, creating a small observation window (not shown) (e.g., about 30-300 nm wide holes). The DNA polymerase 312 can attach complementary nucleotides thus producing multiple copies of complementary sequence. The polymerase 312 can have a height 314, as shown. The above can be similar in concept to a polymerase chain reaction (PCR), as known in the art.

Also present within the trap 142 are free nucleotides or oligonucleotides (e.g., A, C, G, T) and a complementary primer. The nucleotides may or may not be labeled, e.g., with fluorescent tags. FIG. 7A shows a DNA single strand molecule 310 in the trap 142, with nucleotides A, C, G, T identified on the molecule 310. The complementary free nucleotide for A is T; the complementary free nucleotide for C is G; the complementary free nucleotide for G is C (twice); and the complementary free nucleotide for T is A. A complementary nucleotide is attached to the DNA single strand molecule 310, in order, approximately every 1 millisecond to a second, e.g., ˜14 ms, depending on the specific polymerase used.

Still with reference to FIG. 7A, optical sequencing using PCSEL emitter 128 can include tracking fluorescence, including a) time constant(s), b) intensity, and/or c) binding kinetics. The various traps 142 can be located on top of corresponding PCSELs emitters 128, as shown. An example process of DNA/RNA/protein sequencing is also further explained in Reed using dye-labelled NAA recognizers and aminopeptidases for cleaving. These and other aspects and details are contemplated herein.

In more detail, a process using controller 150 includes using the controller 150 to probe the immobilized DNA/RNA/protein molecules 310 in real-time using PCSELs emitters 128 underneath (or otherwise proximate). To probe molecules, the controller 150 can forward bias the PCSEL emitters 128, creating a fluorescence signal (127). See Reed, referenced above, for additional detail and operational details contemplated herein, including details relating to using intensity, time constant and binding kinetics for sequencing, certain concepts of which are applied herein using PCSELs. Embodiments that would utilize NAA or polymerase may depend on what type of molecule is being sequenced, e.g., DNA, RNA, protein, etc. Reed provides additional details for protein and molecular sequencing. For example, NAA can be used to characterize time constant, intensity, and/or binding kinetics for protein sequencing. Reed discusses using aminopeptidases for cleaving. In various optional embodiments, the resulting fluorescent signal 127 can be created by mixing the molecule 310 with dye labelled N-terminal amino acid (NAA) recognizer or polymerase and can optionally be simultaneously cleaved by aminopeptidase. These concepts can be modified as discussed herein for application to PCSELs, such as readings emitted fluorescent signals 127 by PCSELs 128 using the controller 150 by reverse biasing the emitters 128 to act as photodetector, or sensor 128.

A time constant can estimated based on a rate of decay of fluorescence intensity. The time constant can reflect the fluorescence decrease over time at a specific wavelength, and is therefore specific to a given nucleotide. However, a peak intensity itself can depend on the wavelength. An emitter/detector 128 therefore has a particular intensity response for a given wavelength. Therefore, different wavelength signals would produce different corresponding intensity responses. Peak intensity can correspond to a response of a photodetector (emitter/detector 128) at a particular wavelength.

Since a contemplated resulting fluorescent signal 127 has a specific time constant, intensity and binding kinetics, the PCSEL-as-photodetector can characterize the time constant and intensities of the fluorescent signal to recognize the DNA/RNA/protein molecules 310.

Each plasmonic DNA sequence is individually addressed by each photonic laser light delivery (emission) and then detection (sensing), which can beneficially occur sequentially and in parallel using a plurality of PCSELs and corresponding emitters 128 for both emission and sensing, thus greatly simplifying the resulting optical molecular sequencing systems described herein.

Still with reference to FIG. 7A, excitation resulting emission from emitter 128 occur as shown and beam 126 results. The beam 126 can strike the molecule 310 The characteristic emission 127 is then received back at the emitter 128 for analysis and/or sequencing.

The DNA molecule 310 can be labeled with a fluorescent tag (or signature). Labeled molecule 310 analysis procedure details transmitted at emission 127 to emitter/detector 128 are shown at 316. As a result, various colors can be emitted, representing a different nucleotide every time period (˜14 ms) as shown at 318A. Furthermore, different time constants are emitted representing different nucleotide every time period (˜14 ms). In various embodiments, therefore, a spectrometer can be avoided entirely.

FIG. 7A has fluorescence labeled or tagged free nucleotides present in the trap 142, shown as A(dATP), C(dCTP) twice, G(dGTP) and T(dTTP) as shown at 316. As the free nucleotides incorporate into the single DNA strand, a signal is emitted depending on which nucleotide (A(dATP), C(dCTP), G(dGTP), T(dTTP)) is incorporated to the strand, the signal varying by, e.g., intensity of fluorescence, color (wavelength) of fluorescence, or time decay constant (τ1, τ2, τ3, τ4). For example, referring to 316 of FIG. 7A, a blue wavelength or first decay constant (τ1) represents the A nucleotide being added to the strand and thus the original nucleotide being T, a red wavelength or second decay constant (τ2) represents the C nucleotide being added to the strand and thus the original nucleotide being G, a green wavelength or third decay constant (τ3) represents the G nucleotide being added to the strand and thus the original nucleotide being C, and a yellow wavelength or fourth decay constant (τ4) represents the T nucleotide being added to the strand and thus the original nucleotide being A. From the changing signal, the sequence of nucleotides can be determined.

With reference now to the methodology 301 shown in FIG. 7B, if DNA molecule 310 is non-labelled a Raman signature wavelength emission can be observed for different nucleotides every time period 318B (˜1 ms). Non-labeled molecule 310 analysis procedure details as transmitted via output signal 127 are shown at 317. For methodology 301, non-labeled or untagged free nucleotides are present in the trap 142.

Shown in FIG. 7B, as the free nucleotides incorporate into the single DNA strand 310, a signal 127 is emitted depending on which nucleotide (A, C, G, T) is incorporated to the strand 310, the signal varying by, e.g., intensity, wavelength (λ1, λ2, λ3, λ4) or time decay constant (τ1, τ2, τ3, τ4) of the Raman signal emitted, as shown at 317. For example, at 317, a first wavelength (λ1) or first decay constant (τ1) represents the A nucleotide being added to the strand and thus the original nucleotide being T, a second wavelength (λ2) or second decay constant (τ2) represents the C nucleotide being added to the strand and thus the original nucleotide being G, a third wavelength (λ3) or third decay constant (τ3) represents the G nucleotide being added to the strand and thus the original nucleotide being C, and a fourth wavelength (λ4) or fourth decay constant (τ4) represents the T nucleotide being added to the strand and thus the original nucleotide being A. From the changing signal, the sequence of nucleotides (A, C, T, G) can be determined.

More generally, the incorporation of the free nucleotide results in the emittance of a signal depending on which nucleotide (A, C, T, G) is incorporated to the molecule 310. The PCSEL emitter 128 detects the output signal emitted depending on which nucleotide is incorporated to the strand, the signal varying by, e.g., intensity of fluorescence, color (wavelength λ) of fluorescence, or time decay constant (e.g., τ or T) of fluorescence.

With either methodology 300 or 301, light 126 from the PCSEL emitter 128 interacts with the nucleotides (A, C, T, G) of molecule 310 in the trap 142. The PCSEL emitter 128 then detects the signature signal emitted 127 by nucleotides to identify the nucleotides for sequencing.

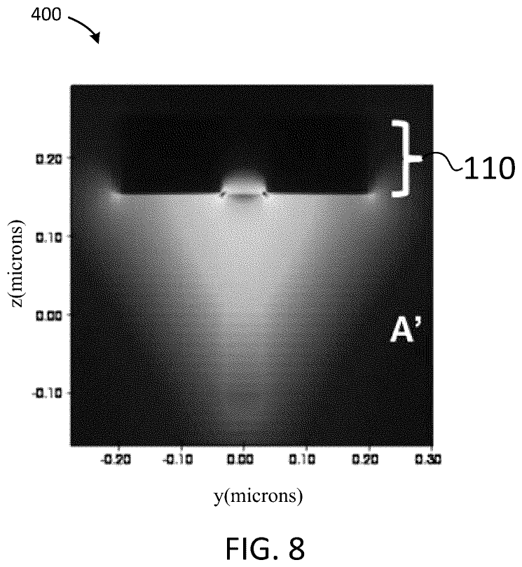

FIG. 8 at view 400 shows strong optical fields at the base of a metal well/trap, according to various embodiments. Plasmonics can play a role in contemplated embodiments such as by enhancing a field at a base of the trap 142 for interaction with DNA molecule 310, at the base of the trap 142 for interaction with DNA sequence.

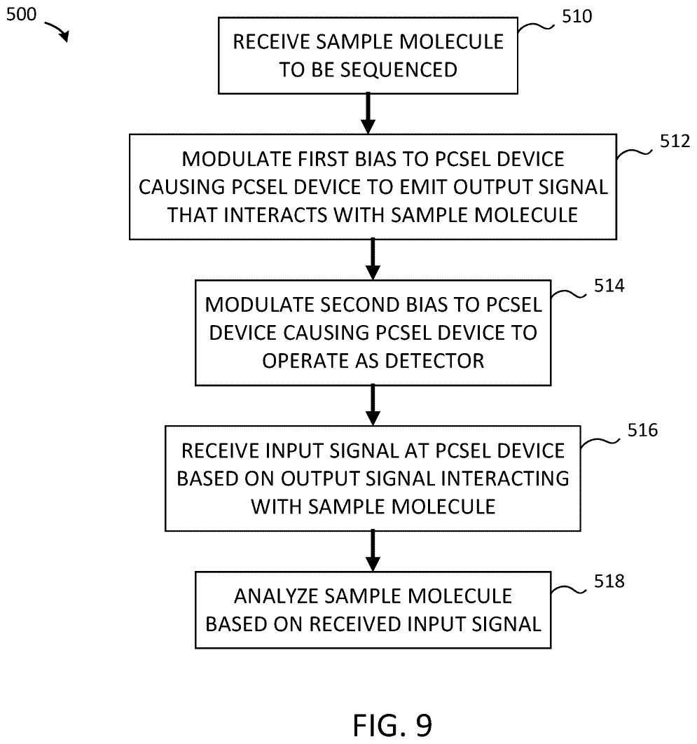

FIG. 9 shows a process 500, according to various embodiments. Process 500 can start by receiving a sample molecule to be sequenced at operation 510. Next, using a controller (e.g., controller 150) a first bias can be modulating to a PCSEL device, causing the PCSEL device to emit an output signal (e.g., beam) that interacts with the sample molecule at operation 512. Following operation 512, a second bias can be modulated to the PCSEL device, causing the PCSEL to operate as a detector, the second bias modulated by the controller at operation 514. Next, at operation 516, an input signal can be received at the PCSEL device based on the output signal interacting with the sample molecule. Then, at operation 518, the controller can analyze the sample molecule based on the input signal received at the PCSEL device.

FIG. 10 is a block schematic diagram of a computer system 600 according to embodiments of the present disclosure. The computer system 600 can be implemented for performing laser-based molecular sequencing, such as using dual-purpose PCSELs emitters/detectors (128), e.g., according to FIGS. 1-9, above.

Computer system 600, as shown, is configured with an interface 616 to enable controller 150 to receive a request to perform laser-based molecular sequencing using PCSELs, as described with regard to FIGS. 1-9. An input 618 may be received at interface 616. In embodiments, the interface 616 can enable controller 150 to receive, or otherwise access, the input 618 via, for example, a network (e.g., an intranet, or a public network such as the Internet), or a storage medium, such as a disk drive internal or connected to controller 150. The interface can be configured for human input or other input devices, such as described later in regard to components of controller 150. It would be apparent to one of skill in the art that the interface can be any of a variety of interface types or mechanisms suitable for a computer, or a program operating in a computer, to receive or otherwise access or receive a source input or file.

Processors 612, 614 included in controller 150 are connected by a memory interface 620 to memory device or module 630. In embodiments, the memory 630 can be a cache memory, a main memory, a flash memory, or a combination of these or other varieties of electronic devices capable of storing information and, optionally, making the information, or locations storing the information within the memory 630, accessible to a processor. Memory 630 can be formed of a single electronic (or, in some embodiments, other technologies such as optical) module or can be formed of a plurality of memory devices. Memory 630, or a memory device (e.g., an electronic packaging of a portion of a memory), can be, for example, one or more silicon dies or chips, or can be a multi-chip module package. Embodiments can organize a memory as a sequence of bit, octets (bytes), words (e.g., a plurality of contiguous or consecutive bytes), or pages (e.g., a plurality of contiguous or consecutive bytes or words).

In embodiments, computer 600 can include a plurality of memory devices. A memory interface, such as 620, between one or more processors 612/614 and one or more memory devices 630 can be, for example, a memory bus common to one or more processors and one or more memory devices. In some embodiments, a memory interface, such as 620, between a processor (e.g., 612, 614) and a memory 630 can be point to point connection between the processor and the memory, and each processor in the computer 600 can have a point-to-point connection to each of one or more of the memory devices. In other embodiments, a processor (for example, 612) can be connected to a memory (e.g., memory 630) by means of a connection (not shown) to another processor (e.g., 614) connected to the memory 630 (e.g., 620 from processor 614 to memory 630).

Computer 600 can include an input/output (I/O) bridge 650, which can be connected to a memory interface 620, or to processors 612, 614. The I/O bridge 650 can interface the processors 612, 614 and/or memory devices 630 of the computer 600 (or, other I/O devices) to I/O devices 660 connected to the bridge 650. For example, controller 150 includes I/O bridge 650 interfacing memory interface 620 (and/or 622) to I/O devices, such as I/O device 660. In some embodiments, an I/O bridge can connect directly to a processor or a memory, or can be a component included in a processor or a memory. An I/O bridge 650 can be, for example, a peripheral component interconnect express (PCI-Express) or other I/O bus bridge, or can be an I/O adapter.

The 1/O bridge 650 can connect to I/O devices 660 by means of an I/O interface, or I/O bus, such as I/O bus 622 of controller 150. For example, I/O bus 622 can be a PCI-Express or other I/O bus. I/O devices 660 can be any of a variety of peripheral I/O devices or I/O adapters connecting to peripheral I/O devices. For example, I/O device 660 can be a graphics card, keyboard or other input device, a hard disk drive (HDD), solid-state drive (SSD) or other storage device, a network interface card (NIC), etc. I/O devices 660 can include an I/O adapter, such as a PCI-Express adapter, that connects components (e.g., processors or memory devices) of the computer 600 to various I/O devices 660 (e.g., disk drives, Ethernet networks, video displays, keyboards, mice, styli, touchscreens, voice control interfaces, etc.).

Computer 600 can include instructions executable by one or more of the processors 612, 614 (or, processing elements, such as threads of a processor). The instructions can be a component of one or more programs. The programs, or the instructions, can be stored in, and/or utilize, one or more memory devices of computer 600. As illustrated in the example of FIG. 6, controller 150 includes a plurality of programs or modules, such as beam module 608, PCSEL module 604, bias modulation module 606, emitter module 609, sequencing module 607, and detector module 605. A program can be, for example, an application program, an operating system (OS) or a function of an OS, or a utility or built-in function of the computer 600. A program can be a hypervisor, and the hypervisor can, for example, manage sharing resources of the computer 600 (e.g., a processor or regions of a memory, or access to an I/O device) among a plurality of programs or OSes.

Programs can be “stand-alone” programs that execute on processors and use memory within the computer 600 directly, without requiring another program to control their execution or their use of resources of the computer 600. For example, controller 150 includes (optionally) stand-alone programs in beam module 608, PCSEL module 604, bias modulation module 606, emitter module 609, sequencing module 607, and/o detector module 605. A stand-alone program can perform particular functions within the computer 600, such as controlling, or interfacing (e.g., access by other programs) an I/O interface or I/O device. A stand-alone program can, for example, manage the operation, or access to, a memory (e.g., memory 630). A basic I/O subsystem (BIOS), or a computer boot program (e.g., a program that can load and initiate execution of other programs) can be a standalone program.

Controller 150 within computer 600 can include one or more OS 602, and an OS 602 can control the execution of other programs such as, for example, to start or stop a program, or to manage resources of the computer 600 used by a program. For example, controller 150 includes OS 602, which can include, or manage execution of, one or more programs, such as OS 602 including (or, managing) beam module 608, PCSEL module 604, bias modulation module 606, emitter module 609, sequencing module 607, and/or detector module 605. In some embodiments, an OS 602 can function as a hypervisor.

A program can be embodied as firmware (e.g., BIOS in a desktop computer, or a hypervisor) and the firmware can execute on one or more processors and, optionally, can use memory, included in the computer 600. Firmware can be stored in a memory (e.g., a flash memory) of the computer 600. For example, controller 150 includes firmware 640 stored in memory 630. In other embodiments, firmware can be embodied as instructions (e.g., comprising a computer program product) on a storage medium (e.g., an optical disc, flash memory, or disk drive), and the computer 600 can access the instructions from the storage medium.

In embodiments of the present disclosure, computer 600 can include instructions for using PCSELs in molecular sequencing applications. Controller 150 includes, for example, beam module 608, PCSEL module 604, bias modulation module 606, emitter module 609, sequencing module 607, and detector module 605, which can operate to provide laser-based molecular sequencing operation using PCSELs, according to various embodiments herein.

The example computer system 600 and controller 150 are not intended to limiting to embodiments. In embodiments, computer system 600 can include a plurality of processors, interfaces, and inputs and can include other elements or components, such as networks, network routers or gateways, storage systems, server computers, virtual computers or virtual computing and/or I/O devices, cloud-computing environments, and so forth. It would be evident to one of skill in the art to include a variety of computing devices interconnected in a variety of manners in a computer system embodying aspects and features of the disclosure.

In embodiments, controller 150 can be, for example, a computing device having a processor (e.g., 612) capable of executing computing instructions and, optionally, a memory 630 in communication with the processor 612. For example, controller 150 can be a desktop or laptop computer; a tablet computer, mobile computing device, personal digital assistant (PDA), tablet, smartphone, or other mobile device; or, a server computer, a high-performance computer (HPC), or a super computer. Controller 150 can optionally be, for example, a computing device incorporated into a wearable apparatus (e.g., an article of clothing, a wristwatch, or eyeglasses), an appliance (e.g., a refrigerator, or a lighting control), a mechanical device, or, e.g., a motorized vehicle. It would be apparent to one skilled in the art that a computer embodying aspects and features of the disclosure can be any of a variety of computing devices having processors and, optionally, memory devices, and/or programs.

Details regarding various methodologies for fluorescence time decay can be found in, e.g., “Development of a Time Domain Fluorimeter for Fluorescent Lifetime Multiplexing Analysis” by Christopher D. Salthouse et al., IEEE Transactions on BioMedical Circuits and Systems, Vol. 2, No. 3, September 2008, and “DNA Sequencing by Capillary Electrophoresis with Four-Decay Fluorescence Detection” by Hui He et al. from the Department of Chemistry at Duke University, Analytical Chemistry, Vol. 72, pp. 5865-5873, 2000. He et al. provides further contemplated examples directed to utilizing fluorescence labeled free nucleotides and how the outgoing wavelength or time decay constant can be used to identify nucleotides. See also, “Bright Unidirectional Fluorescence Emission of Molecules in a Nanoaperture with Plasmonic Corrugations” by Heykel Aouani et al., Wiley Interdisciplinary Reviews Nanomedicine and Nanobiotechnology, May 2014.

Applicant's co-pending U.S. application Ser. No. 18/603,512, filed Mar. 13, 2024, is hereby incorporated by reference for all purposes herein.

The present invention has now been described with reference to several embodiments thereof. The foregoing detailed description and examples have been given for clarity of understanding only. No unnecessary limitations are to be understood therefrom. It will be apparent to those skilled in the art that many changes can be made in the embodiments described without departing from the scope of the invention. The implementations described above and other implementations are within the scope of the following claims.

What is claimed is:

1. A laser-based molecular sequencing system, comprising:
a two-dimensional photonic crystal surface emitting laser (PCSEL) array comprising a plurality of PCSEL devices located in a first layer, each PCSEL device oriented in a direction perpendicular to the first layer; and
a controller configured to modulate a bias on at least one PCSEL device of the plurality of PCSEL devices, the modulation configured to selectively operate each PCSEL device as an emitter or detector at a point in time based on the bias.

2. The system of claim 1, wherein the controller is further configured to:
receive a communication from a first PCSEL device biased to operate as a detector, and
analyze a sample molecule located proximate the first PCSEL device.

3. The system of claim 2, wherein the analyzing comprises detecting at least one of a:
a) phase,
b) amplitude, or
c) time-based aspect received at the first PCSEL device.

4. The system of claim 3, wherein the received aspect is a signal emitted by the sample molecule.

5. The system of claim 2, wherein the sample molecule comprises DNA, RNA, or protein.

6. The system of claim 2, wherein the analyzing comprises sequencing or categorizing nucleotides of the sample molecule.

7. The system of claim 6, wherein the sample molecule comprises a DNA polymerase in a molecule trap, and wherein a nucleotide is identified based on a spectral signature of the sample molecule.

8. The system of claim 1, wherein the plurality of PCSEL devices form a preset pattern within the first layer.

9. The system of claim 1, further comprising a plurality of molecule traps.

10. The system of claim 9, wherein the plurality of molecule traps form an array that corresponds to the array of PCSEL devices.

11. The system of claim 1, wherein the plurality of PCSEL devices are integrated with a semiconductor chip.

12. The system of claim 1, wherein the PCSEL devices are configured function as a spectrometer and optionally filter aspects.

13. The system of claim 12, wherein the PCSEL devices are further configured to identify a spectral signature.

14. The system of claim 13, wherein the identifying is based at least in part of a wavelength or fluorescence corresponding to a spectral signature associated with a nucleotide of a molecule.

15. The system of claim 14, wherein the nucleotide is labeled or non-labeled, and the spectral signature comprises fluorescent and/or Raman-based signals.

16. The system of claim 14, wherein the spectral signature comprises a wavelength and/or time-decay constant.

17. A method of performing laser-based molecular sequencing, comprising:
receiving a sample molecule to be sequenced;
modulating a first bias to a PCSEL device to cause the PCSEL device to emit an output signal, wherein the output signal interacts with the sample molecule;
modulating a second bias to the PCSEL device to cause the PCSEL device to operate as a detector;
receiving an input signal at the PCSEL device based on the output signal interacting with the sample molecule; and
analyzing the sample molecule based on the input signal.

18. The method of claim 17, wherein the sample molecule comprises DNA, RNA, or protein, and wherein the analyzing comprises sequencing nucleotides of the sample molecule.

19. A computer program product for performing laser-based molecular sequencing, the computer program product comprising a computer-readable storage medium having program code embodied therewith, the program code comprising computer-readable program code configured to cause a processor to perform the steps of:
receiving a sample molecule to be sequenced;
modulating a first bias to a PCSEL device to cause the PCSEL device to emit an output signal, wherein the output signal interacts with the sample molecule;
modulating a second bias to the PCSEL device to cause the PCSEL device to operate as a detector;
receiving an input signal at the PCSEL device based on the output signal interacting with the sample molecule; and
analyzing the sample molecule based on the input signal.

20. The computer program product of claim 19, wherein the sample molecule comprises DNA, RNA, or protein, and wherein the analyzing comprises sequencing nucleotides of the sample molecule.
